(19) United States
(12) Patent Application Publication
(10) Pub. No.: US 2011/0028939 A1
(43) Pub. Date: Feb. 3, 2011

(54) TIP END ASSEMBLY

(75) Inventor: Richard J. Yarger, Yakima, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: SURGIMARK, INC., Yakima, WA (US)

(21) Appl. No.: 12/533,512

(22) Filed: Jul. 31, 2009

(51) Int. Cl. A61M 1/00

(52) U.S. Cl. 604/523

(57) ABSTRACT

A tip end assembly (10) for a surgical aspirator tip (14) is provided. The surgical aspirator tip (14) includes a cannula (18) with a distal end opening (24), wherein the cannula (18) extends from and is in fluid flow communication with a hollow handle (22) configured to be placed into fluid flow communication with a suction source. The tip end assembly (10) is comprised of a flared end (68) defined on the distal end of the cannula (18) and a hollow tip guard (46) having at least one opening (52, 54). The tip guard (46) is molded onto the flared end (68) of the cannula (18) such that the at least one opening (52, 54) of the tip guard (46) is in fluid flow communication with the distal end opening (24) of the cannula (18).

### BACKGROUND

Surgical aspirators are used to remove fluids from the body of the patient. A surgical aspirator typically includes an elongated aspirator tip that is inserted into a surgical site, wound, or other bodily orifice. The aspirator tip is typically comprised of a hollow handle to facilitate using and holding the aspirator and a stainless steel cannula extending from the handle and in fluid communication therewith. The proximal end of the handle is connectable to a suction tube which in turn is connected to a suction pump that provides suction to the aspirator tip. The cannula includes a tip end opening for drawing fluids, gases, and other materials into the aspirator tip when inserted within a surgical site, wound, bodily orifice, etc. A tip guard is normally molded onto the distal end of the cannula that includes a tip guard end opening and several cross-holes in fluid communication with the tip end opening of the cannula.

Pieces of tissue and other debris may be suspended in the fluids and can clog the openings in the tip guard molded onto the distal end of the cannula. Various improvements in aspirator tip design have been made to help prevent the tip guard and aspirator tip from becoming clogged. For instance, the cross-holes in the tip guard may be formed within grooves defined between axial ridges, wherein the ridges help prevent the tissue from reaching the cross-holes while allowing fluid to be evacuated through the cross-holes.

The distal end of the aspirator tip may also be covered with a sleeve that is formed with a plurality of small holes. The sleeve prevents the tissue from reaching the tip guard while allowing the fluid being evacuated to flow into the sleeve through the holes. The sleeve may also include internal projections defined on its interior surface to maintain the position of the sleeve relative to the aspirator tip and help ensure adequate space between the aspirator tip and the sleeve. Therefore, fluids and small debris may flow freely to or through the aspirator tip end opening.

Venting channels may additionally be formed between the sleeve and tip to sustain uniform distribution of suction in the event that the holes in the sleeve become clogged. To effectively distribute the suction, the venting channels should be properly aligned with the sleeve to ensure that airflow reaches the interior of the sleeve if any of the holes become clogged. A sleeve locking mechanism may be used to secure the position of the sleeve relative to the aspirator tip such that the venting channels are maintained between the sleeve and tip during use.

The above-described embodiments of a surgical aspirator tip and sleeve assembly are described in U.S. patent application Ser. No. 11/736,396, filed Apr. 17, 2007; U.S. patent application Ser. No. 11/405,270, filed Apr. 14, 2006; U.S. patent application Ser. No. 11/303,702, filed Dec. 15, 2005; U.S. patent application Ser. No. 10/969,276, filed Oct. 19, 2004; U.S. patent application Ser. No. 10/153,420, filed May 22, 2002; the disclosures all of which are hereby expressly incorporated herein by reference.

As mentioned above, the proximal end of the tip is connected to a suction tube that is in communication with a suction pump to provide suction to the tip. Even if the aspirator tip guard does not become clogged with the tissue and other debris due to the improved designs discussed above, the suction tube may become clogged. In an effort to unclog or clear the suction tube, the surgeon or surgical assistant will often detach the aspirator tip from the tube and then shove or push a separate wire or other elongated device into the suction tube (“the clearing process”). After clearing the tube with the wire, the wire may become lost, dropped, etc. In an effort to consolidate parts and minimize waste, the surgeon or surgical assistant may instead shove or push the distal end of the cannula, including the tip guard, into the suction tube during the clearing process. However, during this process, the tip guard may detach from the distal end of the cannula, rendering the aspirator tip unusable.

Currently known methods of securing the tip guard to the cannula do not effectively prevent the detachment of the tip guard from the cannula during the clearing process. Such methods include, for instance, drilling cross-holes into the distal end of the cannula for receiving plastic during the molding process to define projections secured within the cannula. However, these projections often shear off during the clearing process. Other methods include roughening the distal end of the cannula on which the tip guard is molded in an attempt to increase the friction between the cannula and the tip guard or using a bonding agent to secure the tip guard to the cannula. Neither of these methods prevents the tip guard from detaching and sliding axially off the end of the cannula or moving axially along the cannula.

None of the above-described tip guard assemblies or methods of securing a tip guard to the distal end of a cannula effectively prevent the tip guard from detaching from the cannula during the clearing process. As such, a need exists for an improved assembly and method of securing a tip guard to a distal end of a cannula.

### SUMMARY

A tip end assembly for a surgical aspirator tip is provided. The surgical aspirator tip includes a cannula with a distal end opening, wherein the cannula extends from and is in fluid flow communication with a hollow handle configured to be placed into fluid flow communication with a suction source. The tip end assembly is comprised of a flared end formed on the distal end of the cannula and a hollow tip guard having at least one opening. The tip guard is molded directly onto the flared end of the cannula such that the at least one opening of the tip guard is in fluid flow communication with the distal end opening of the cannula.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the present disclosure will become more readily appreciated by reference to the following detailed description, when taken in conjunction with the accompanying drawings, wherein:

FIG. 1 is an isometric view of a preferred embodiment of a tip end assembly of a surgical aspirator tip;

FIG. 2 is an isometric view of a portion of the tip end assembly of FIG. 1;

FIG. 3 is a cross-sectional view of the tip end assembly of FIG. 1; and

FIG. 4 is a cross-sectional view of an alternate embodiment of a tip end assembly.

### DETAILED DESCRIPTION

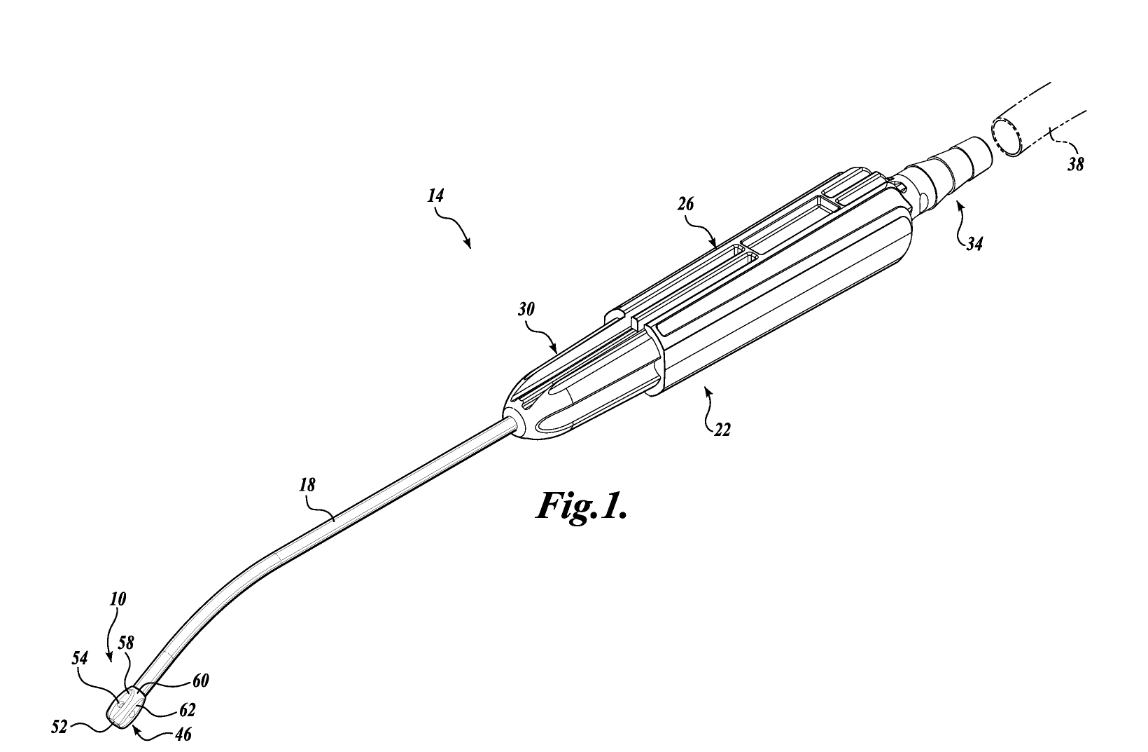

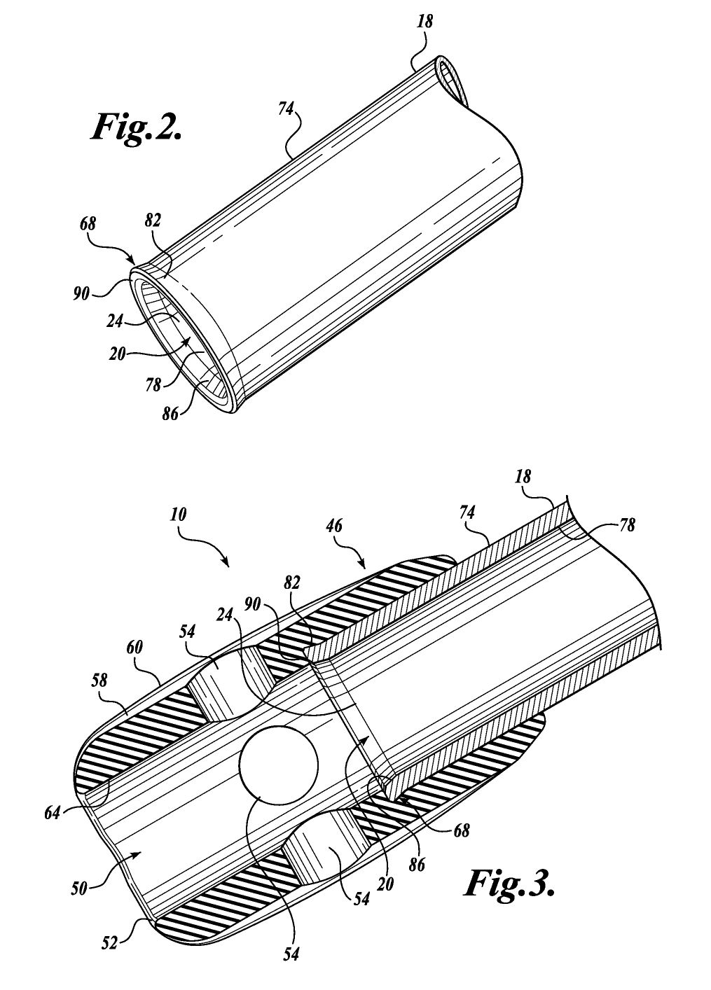

Referring to FIG. 1, a tip end assembly 10 formed in accordance with a preferred embodiment of the present disclosure is depicted as a portion of a surgical aspirator tip 14. The illustrated surgical aspirator tip 14 is a Yankauer or Andrews aspirator tip having a hollow tubular neck member, or cannula 18 that is secured to and in fluid communication with an elongated, hollow handle 22. The cannula 18 includes an inner passageway 20 and extends from a distal end of the handle 22 such that it is configured to be inserted into a wound, bodily orifice, surgical site, etc., for removal of fluids. The cannula 18 may include a slight bend, as shown, or it may instead be substantially straight and extend axially from the handle 22. The distal end of the cannula 18 defines an end opening 24 (see FIG. 2) into which gases, fluids, and materials can flow. The cannula 18 is preferably constructed from a suitable metal, such as stainless steel.

The handle 22 is secured to the cannula 18 in a manner well known in the art and is preferably constructed from a rigid or semi-rigid, resiliently deformable material that is adaptable for use in the medical arts, such as a polymeric or resinous plastic. The handle 22 includes a grip section 26 that is sized and shaped for gripping the aspirator tip 14, a male coupling section 30 that is sized and configured to frictionally receive an aspirator sleeve thereon (not shown), and a tube coupling section 34 that is sized and configured to frictionally receive a suction tube 38 thereon. The suction tube 38 is in turn connected to a source of suction (not shown), such as a suction pump, to provide suction to the aspirator tip 14. In this manner, suction supplied to the tube 38 flows through the handle 22, into the cannula 18, and through the end opening 24 of the cannula 18.

Although the tip end assembly 10 will be hereinafter described and illustrated generally as a portion of a Yankauer or Andrews aspirator tip 14, one skilled in the relevant art will appreciate that the disclosed embodiment may be used on any similar aspirator tip, such as a Frazier aspirator tip or an aspirator tip having any combination of features described generally above. Moreover, although the illustrated aspirator tip 14 is shown and described without reference to an aspirator sleeve, it should be appreciated that the aspirator tip 14 having the tip end assembly 10 may be used with any suitable aspirator sleeve. It should therefore be apparent that the disclosed embodiment of the tip end assembly 10 has wide application and may be used on any similar aspirator tip or aspirator tip and sleeve combination. Accordingly, the following descriptions and illustrations herein should be considered illustrative in nature, and thus not limiting the scope of the present disclosure, as claimed.

Referring to FIGS. 1-3, the tip end assembly 10 will be hereinafter described in detail. The tip end assembly 10 is comprised of a hollow tip guard 46 molded onto a distal end of the cannula 18 in a manner that prevents the tip guard 46 from detaching from the cannula 18 during the clearing process. Although it should be appreciated that any suitable tip guard may be secured to the cannula 18, the tip guard 46 preferably includes a contoured outer surface 60 to help prevent damage to the tissue at the surgical site when engaging the tissue to aspirate fluids, debris, etc. The illustrated tip guard 46 further includes an inner passageway 50 defined by a substantially cylindrical tip guard inner surface 64 and a tip guard end opening 52 formed in the distal end of the tip guard 46 that is in communication with the inner passageway 50. The inner passageway 50 is in fluid communication with the inner passageway 20 of the cannula 18 when the tip guard 46 is mated to the cannula 18. In this manner, gases, fluids, materials, etc. may easily flow into the tip guard 46 and thereafter into the cannula 18.

The tip guard 46 also preferably includes a plurality of cross-holes 54 in fluid communication with the inner passageway 50 to help prevent clogging of the aspirator tip 14 during use. The cross-holes 54 may be arranged on the tip guard 46 in any suitable manner; however, the cross-holes 54 are preferably spaced equidistant from one another circumferentially around the tip guard 46. The cross-holes 54 are also preferably formed within axial grooves 58 extending along at least a portion of the tip guard 46. The axial grooves 58 extend inwardly from the tip guard outer surface 60 to effectively define ridges 62 in between each pair of adjacent cross-holes 54. The ridges 62 engage the tissue during aspiration to help prevent the tissue from reaching the cross-holes 54 such that fluid may be evacuated through the cross-holes 54.

During aspiration, gases, fluids, and materials may flow into the end opening 52 and cross-holes 54 of the tip guard 46, through the end opening 24 of the cannula 18 and into the inner passageway 20 of the cannula 18, and through the handle 22 and into the suction tube 38. It should be appreciated that any other suitable arrangement of openings may be formed within the tip guard 46 to effectively aspirate the wound, surgical site, etc., without clogging the aspirator tip 14.

Referring to FIG. 2, the tip end assembly 10 further includes a flared end 68 formed on the distal end of the cannula 18. The flared end 68 is defined by a predetermined axial length of the cannula 18 that extends radially outwardly from the cannula outer surface 74 at a predetermined angle from the center longitudinal axis of the cannula 18. The axial length of the cannula 18 forming the flared end 68 will vary to accommodate tip guards of various lengths and thicknesses being molded onto the distal end of the cannula 18. More specifically, the flared end 68 should be formed from a suitable axial length such that the flared end 68 extends into a portion of the body of the tip guard 46 when the tip guard 46 is molded thereto.

The flared end 68 also preferably extends radially outwardly from the center longitudinal axis of the cannula 18 at an acute angle to extend into a portion of the body of the tip guard 46 and prevent the tip guard 46 from becoming detached from the cannula 18 during the clearing process or during another similar process. The flared end 68 may extend radially outwardly from the center longitudinal axis of the cannula 18 at any suitable angle, such as, for instance, a fifteen degree (15°) angle. However, it should be appreciated that the flared end 68 may instead extend from the cannula 18 at an angle within a suitable range, such as, for example, at an angle in between five degrees (5°) and ninety degrees (90°).

The flared end 68 defines a flared end outer surface 82 that extends from the cannula outer surface 74 at the predetermined angle (i.e., at a 15° angle or at any suitable angle within a predetermined range), a flared end inner surface 86 that extends from the cannula inner surface 78, and an end surface 90 that extends substantially transversely from the inner surface 86 to the outer surface 82 or at any suitable angle. The flared end inner surface 86 may extend from the cannula inner surface 78 at any suitable predetermined angle or instead at a substantially zero degree (0°) angle such that the flared end inner surface 86 is effectively a continuous extension of the cannula inner surface 78. Preferably, the flared end inner surface 86 is substantially parallel to the flared end outer surface 82 and extends from the cannula inner surface 78 at substantially the same angle as the flared end outer surface 82 (i.e., at about a 15° angle or at any suitable angle within a predetermined range). In this manner, the flared end 68 may be formed by a suitable manufacturing process, such as by placing the end of the cannula 18 over a radially expanding mandrel and then hydraulically or otherwise gradually expanding the diameter of the end of the cannula 18 to create the flared end 68. Alternatively, a roller tool can be used to roll against the cannula inner surface 78 after the cannula 18 is placed in a jig or other tooling fixture. It should be appreciated that any other suitable process for manufacturing a flared end of a stainless steel cannula (or a cannula of another appropriate material) may instead be used.

Although the tip end assembly 10 is described as having a flared end 68 formed by radially expanding a portion of the distal end of the cannula 18, it should be appreciated that the tip end assembly 10 may instead include an annular ring, protrusion, etc., secured to the distal end of the cannula 18 by welding, brazing, or any other suitable method. Moreover, the cannula 18 may instead include an enlarged end portion having any suitable cross-sectional shape that has a nominal outer diameter greater than the nominal outer diameter of the cannula 18 such that the end portion extends into a portion of the tip guard 46 when the tip guard 46 is molded to the cannula 18. Thus, the foregoing description should not be seen as limiting the scope of the claimed subject matter.

Referring to FIG. 3, the tip guard 46 is molded onto the flared end 68 of the cannula 18 to define a tip end assembly 10 of the surgical aspirator tip 14. The tip guard 46 may be molded onto the distal end of the cannula 18 in any suitable manner well known in the art. As a non-limiting example, the tip guard 46 may be formed on the distal end of the cannula 18 by injection molding. It should be appreciated that the handle 22 may be molded onto the proximal end of the cannula 18 using the same or a different molding process; however, for the sake of brevity, only the method by which the tip guard 46 may be molded onto the cannula 18 will be hereinafter described.

To form the tip guard 46, a removable tip guard core (not shown) suitable in diameter for forming the inner passageway 50 of the tip guard 46 may be inserted into the inner passageway 20 of the distal end of the cannula 18. The tip guard core and the cannula 18 may then be placed into a tip guard mold (not shown) having an upper and a lower portion, wherein each portion of the tip guard mold contains a portion of a mold cavity. The mold includes inwardly extending projections that extend into the mold cavity to form the end opening 52 and the cross-holes 54 in the tip guard 46.

Both portions of the tip guard mold are coupled together to define the mold cavity therebetween. At least one inlet channel is included in the mold to allow the inflow of material into the mold cavity. An injection nozzle may inject material through the inlet channel and into the mold cavity. The injected material fills the mold cavity and surrounds a section of the tip guard core and the distal end of the cannula 18, including the cannula flared end 68.

After the material injected into the mold cavity has cured, the mold portions are separated and the cannula 18 and newly formed tip guard 46 are removed from the mold. Next, the tip guard core may be removed from the distal end of the tip guard 46.

The tip guard core may be composed of core materials known in the art such as hardened tool steel or other suitable core material. Similarly, the molds may be constructed from materials known in the art such as hardened tool steel or other suitable mold material. The material injected into the mold to form the tip guard 46 may include polymeric or resinous plastics or any other material suitable for the medical arts.

As shown in FIG. 3, the tip guard 46 is molded onto the distal end of the cannula 18 such that the cannula flared end 68 extends at least partially into the body of the tip guard 46. More specifically, the flared end 68 is of a predetermined angle and defined by a predetermined axial length of the cannula 18 such that the flared end 68 extends into a portion of the body of the tip guard 46; however, the flared end 68 does not protrude through the tip guard outer surface 60. In this manner, the flared end 68 is secured within the molded tip guard 46 but does not protrude therefrom to cause damage to tissue when aspirating fluids. It should be appreciated that the angle and axial length of the flared end 68 will vary depending on the size of the tip guard 46.

Preferably, the diameter of the inner passageway 20 of the cannula 18 is slightly smaller than the diameter of the inner passageway 50 of the tip guard 46. As such, the flared end inner surface 86 extends from the cannula inner surface 78 to the tip guard inner surface 64 to define a substantially smooth transition between the interior of the cannula 18 and the inner passageway 50 of the tip guard 46.

With the flared end 68 of the cannula 18 extending at least partially into the body of the tip guard 46, the tip guard 46 is prevented from detaching during the clearing process. For instance, when an axial force is exerted onto the tip guard 46 in the direction of the cannula 18, the body of the tip guard 46 will abut against the end surface 90 of the flared end 68 to prevent the tip guard 46 from detaching from the cannula 18 and sliding axially along the length of the cannula 18. Such a force may be exerted on the tip guard 46, for example, when pushing or shoving debris or other material through the suction tube 38 during the clearing process.

Moreover, with the flared end 68 being integrally formed as a portion of the stainless steel cannula 18, the flared end 68 will not shear off from the cannula 18 when a force is exerted onto the flared end 68 through the tip guard 46, thereby preventing the detachment of the tip guard 46 from the cannula 18. As such, the flared end 68 also effectively prevents the tip guard 46 from being pulled off of the cannula 18 when an axial pulling force is exerted on the tip guard 46. Such an axial pulling force may be exerted on the tip guard 46 when, for instance, the tip guard 46 and cannula 18 are being removed from the suction tube 38 during or after the clearing process. The body of the tip guard 46 abuts against the flared end outer surface 82 to prevent detachment of the tip guard 46.

If, during the clearing process, the tip guard 46 loosens or detaches from the cannula outer surface 74 and the flared end outer surface 82, the tip guard 46 will remain attached to the cannula 18 and continue to work effectively. More specifically, the tip guard 46 may loosen such that it can rotate relative to the cannula 18; however, the flared end 68 will prevent the tip guard 46 from moving axially along the cannula 18. By maintaining the axial position of the tip guard 46 on the cannula 18, the cross-holes 54 will remain in fluid communication with the inner passageway 20 of the cannula 18 and the tip guard 46 will continue to effectively prevent clogging of the aspirator tip 14 and allow fluids, gases, etc., to flow into the cannula 18. Accordingly, the flared end 68 of the cannula 18 prevents the tip guard 46 from moving axially along the cannula 18 during the clearing process such that the aspirator tip 14 may be re-used for another application.

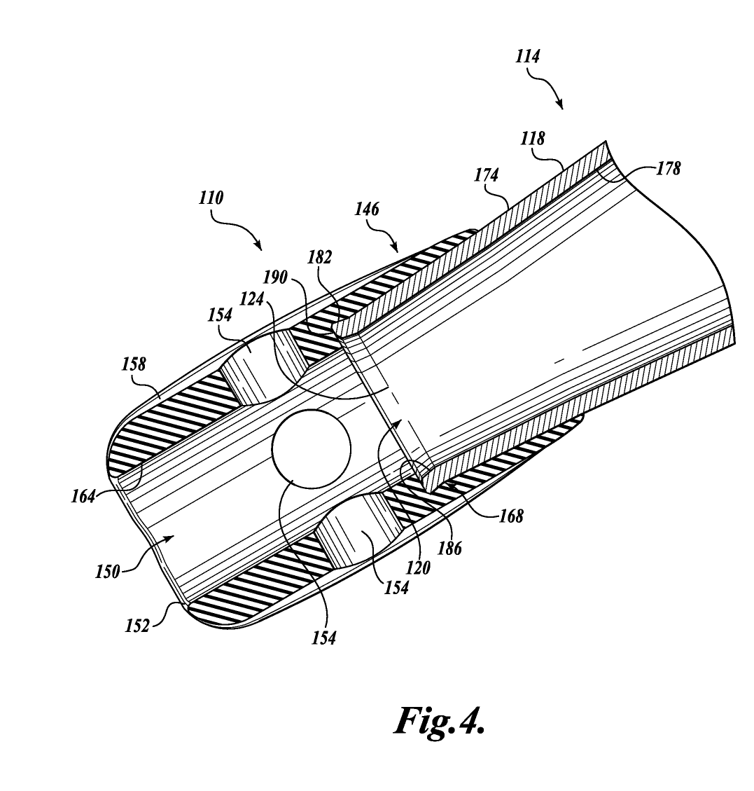

FIG. 4 depicts an alternate embodiment of a tip end assembly 100 substantially similar to the tip end assembly 10 described above except for the differences hereinafter provided. Moreover, it should be noted that at least the same variations and changes may be made to the tip end assembly 100 as those described above with reference to the tip end assembly 10.

It was noted above that the tip end assembly 10, although described as a portion of a Yankauer or Andrews aspirator tip 14, may instead be adapted for use on any similar aspirator tip. FIG. 4 illustrates the tip end assembly 100 incorporated onto the tip end of a well-known Frazier aspirator tip 114 or similar tip. More specifically, the aspirator tip 114 includes a cannula 118 having inner and outer surfaces 178 and 174, wherein the cannula 118 is tapered along its length as it extends from the proximal handle end (not shown) toward the distal end opening 124 of the cannula 118. In other words, the cross-sectional diameter of the cannula 118 is greatest at the proximal end of the cannula 118 and gradually decreases in size with the smallest cross-sectional diameter being defined at the distal end opening 124 of the cannula 118. The tapered shape of the cannula 118 defines a cannula inner passageway 120 that gradually increases in diameter along its length. This tapered shape helps prevent clogging of material within the cannula 118 since material that passes through the smaller distal end opening 124 also typically passes through the remainder of the cannula 118 having a larger cross-sectional shape.

With at least this purpose in mind, a tip guard 146 is molded onto a flared end 168 of the tapered cannula 118 in a substantially similar manner to that described above to prevent the tip guard 146 from detaching from the cannula 118 during the clearing process. As can be seen in FIG. 4, the tip guard 146 is substantially identical in shape, size, and geometry to the tip guard 46 shown in FIG. 3. The tip guard 146 includes an inner passageway 150 defined by a substantially cylindrical tip guard inner surface 164 and a tip guard end opening 152 formed in the distal end of the tip guard 146 that is in communication with the inner passageway 150. The inner passageway 150 is in fluid communication with the inner passageway 120 of the cannula 118 when the tip guard 146 is mated to the cannula 118. In this manner, gases, fluids, materials, etc., may easily flow into the tip guard 146 and thereafter into the cannula 118. The tip guard 146 may further include cross-holes 154 formed within axial grooves 158 to help aspirate fluids and prevent clogging of the aspirator tip 114 during use.

As can also be seen in FIG. 4, the flared end 168 of the cannula 118 extends radially outwardly from the center longitudinal axis of the cannula 118 at a predetermined acute angle to extend into a portion of the tip guard 146, similar to the flared end 68 of cannula 18 (see FIG. 3). The flared end 168 defines a flared end outer surface 182 that extends from the cannula outer surface 174 at the predetermined acute angle, a flared end inner surface 186, and an end surface 190 that extends substantially transversely from the inner surface 186 to the outer surface 182 or at any suitable angle. The flared end inner surface 186 may extend from the cannula inner surface 178 at any suitable predetermined angle or instead at a substantially zero degree (0°) angle such that the flared end inner surface 186 is effectively a continuous extension of the cannula inner surface 178.

In any case, it is preferred that the distal end opening 124 of the cannula 118 be at least somewhat larger in diameter than the inner passageway 150 of the tip guard 146. In this manner, material that passes through the inner passageway 150 of the tip guard 146 will also typically pass into the inner passageway 120 of cannula 118 having a larger diameter. It should be appreciated that the inner passageway 150 of the tip guard 146 may be substantially constant in diameter, as shown, or the inner passageway 150 may instead gradually increase in diameter from the tip guard end opening 152 to the junction of the tip guard 146 and the cannula 118. In this manner, any material passing into the tip end opening 152 should continue to travel through the inner passageway 150 of the tip guard 146 and into the inner passageway 120 of the cannula 118. However, for ease of manufacturing, it should be appreciated that the tip guard 146 is preferably formed with an inner passageway 150 of substantially constant diameter. In this manner, a tip guard core (not shown) of constant cross-sectional diameter may be used to define the inner passageway 150 of the tip guard 146 during the injection molding process or other suitable process.

Based on the foregoing, and further in light of the description provided above with respect to the tip end assembly 10, it can be understood that the flared end 168 of the tapered cannula 118 prevents the tip guard 146 from detaching during the clearing process. It should be appreciated that the tip end assembly may be similarly modified or adapted for use with other similar aspirator tips. While illustrative embodiments have been illustrated and described, it will be appreciated that various changes can be made therein without departing from the spirit and scope of the present disclosure.

1. A tip end assembly for a surgical aspirator tip having a metal cannula with a distal end opening, the metal cannula extending from and in fluid flow communication with a hollow handle configured to be placed into fluid flow communication with a suction source, the tip end assembly comprising:
(a) a flared end defined on the distal end of the metal cannula; and
(b) a hollow plastic tip guard having at least one opening, the plastic tip guard molded onto the flared end of the metal cannula such that the at least one opening of the plastic tip guard is in fluid flow communication with the distal end opening of the metal cannula.

2. The tip end assembly of claim 1, wherein the flared end extends radially outwardly from a center longitudinal axis of the metal cannula at a predetermined angle sufficient to prevent substantial axial movement of the plastic tip guard relative to the metal cannula upon application of an axial force.

3. The tip end assembly of claim 2, wherein the predetermined angle is from five degrees to ninety degrees.

4. (canceled)

5. The tip end assembly of claim 2, wherein the flared end is of a predetermined angle and defined by a predetermined axial length, of the metal cannula such that the flared end extends into a portion of the plastic tip guard without protruding from an exterior surface of the plastic tip guard.

6. The tip end assembly of claim 2, wherein the flared end defines a flared end outer surface, a flared end inner surface, and an end surface extending between the flared end outer surface and the flared end inner surface.

7. The tip end assembly of claim 6, wherein a portion of the plastic tip guard engages the end surface of the flared end when an axial force is exerted on the plastic tip guard.

8. The tip end assembly of claim 6, wherein the flared end inner surface is substantially parallel to the flared end outer surface.

9. The tip end assembly of claim 8, wherein the flared end inner surface extends substantially between an inner surface of the metal cannula and an inner surface of the plastic tip guard.

10. The tip end assembly of claim 1, wherein the metal cannula increases in cross-sectional diameter from the distal end opening to the handle.

11. The tip end assembly of claim 10, wherein the plastic tip guard defines an inner passageway in communication with the at least one opening, wherein the inner passageway is smaller in diameter that the distal end opening of the metal cannula.

12. A surgical aspirator tip having a metal cannula with a distal end opening, the metal cannula extending from and in fluid flow communication with a manually graspable handle configured to be placed into fluid flow communication with a suction source, the surgical aspirator tip comprising:
(a) an end portion formed on the distal end of the metal cannula having an nominal outer diameter greater than the nominal outer diameter of the metal cannula; and
(b) a plastic tip guard having at least one opening, the plastic tip guard molded in place onto the end portion of the metal cannula such that the at least one opening of the plastic tip guard is in fluid communication with the distal end opening of the metal cannula.

13. The surgical aspirator tip of claim 12, wherein the end portion of the metal cannula is defined by a flared end that extends radially outwardly from a center longitudinal axis of the metal cannula at a predetermined angle sufficient to prevent substantial axial movement of the plastic tip guard relative to the metal cannula upon application of an axial force.

14. The surgical aspirator tip of claim 13, wherein the predetermined angle is from five degrees to ninety degrees.

15. The surgical aspirator tip of claim 13, wherein the flared end defines a flared end outer surface, a flared end inner surface, and an end surface extending between the flared end outer surface and the flared end inner surface.

16. The surgical aspirator tip of claim 13, wherein the flared end inner surface is substantially parallel to the flared end outer surface.

17. The surgical aspirator tip of claim 13, wherein the flared end inner surface extends substantially between an inner surface of the metal cannula and an inner surface of the plastic tip guard.

18. The surgical aspirator tip of claim 12, wherein a portion of the plastic tip guard engages the end portion of the metal cannula when an axial force is exerted on the plastic tip guard.

19. The surgical aspirator tip of claim 14, wherein the end portion is of a predetermined axial length and thickness such that the end portion extends into a portion of the plastic tip guard without protruding from the plastic tip guard.

20. The surgical aspirator tip of claim 1, wherein the metal cannula increases in cross-sectional diameter from the distal end opening to the handle.

21. The surgical aspirator tip of claim 20, wherein the plastic tip guard defines an inner passageway in communication with the at least one opening, wherein the inner passageway is smaller in diameter that the distal end opening of the metal cannula.

22. A surgical aspirator tip, comprising:
(a) a manually graspable handle configured to be placed into fluid flow communication with a suction source;
(b) a cannula with a distal end opening, the cannula extending from and in fluid flow communication with the handle;
(c) an enlarged end portion formed on the distal end of the cannula having an nominal outer diameter greater than the nominal outer diameter of the cannula; and
(d) a tip guard having at least one opening, the tip guard molded in place onto the exterior of the enlarged end portion of the cannula and at least a portion of the distal end of the cannula such that the at least one opening of the tip guard is in fluid communication with the distal end opening of the cannula, wherein the enlarged end portion has a predetermined nominal outer diameter such that the enlarged end portion extends into a portion of the tip guard without protruding from an exterior surface of the tip guard to substantially prevent axial movement of the tip guard along the cannula.
